(19) United States
(12) Patent Application Publication
(10) Pub. No.: US 2022/0001650 A1
(43) Pub. Date: Jan. 6, 2022

(54) METHOD OF ADHESIVE SELECTION FOR COLD FORMING PRODUCT AND PROCESS

(71) Applicant: CORNING INCORPORATED, CORNING, NY (US)

(72) Inventors: Gaurav Dave, Painted Post, NY (US); Rohan Ram Galgalikar, Painted Post, NY (US); Khaled Layouni, Fontainebleau (FR); Arpita Mitra, Columbus, OH (US); Wei Xu, Horseheads, NY (US)

(21) Appl. No.: 17/285,722

(22) PCT Filed: Oct. 18, 2019
(86) PCT No.: PCT/US2019/056929

§ 371 Date: Apr. 15, 2021

Related U.S. Application Data
(60) Provisional application No. 62/814,906, filed on Mar. 7, 2019.
(60) Provisional application No. 62/755,203, filed on Nov. 2, 2018.
(60) Provisional application No. 62/747,531, filed on Oct. 18, 2018.

(51) Int. Cl. B32B 17/06; B32B 7/12; B32B 7/027; B32B 15/18; B32B 15/20; C03C 27/04; C09J 175/04; C09J 183/04; C09J 163/00; G01N 19/04; G01N 3/24

(52) U.S. Cl. CPC B32B 17/061; G01N 2203/006; B32B 7/027; B32B 15/18; B32B 15/20; C03C 27/048; C09J 175/04; C09J 183/04; C09J 163/00; G01N 19/04; G01N 3/24; B32B 2605/003; B32B 2255/00; B32B 2307/4023; G01N 2203/0017; G01N 2203/0025; G01N 2203/0075; B32B 7/12

(57) ABSTRACT

Aspects of this disclosure relate to a method for selecting an adhesive for bonding a cold-formed glass to a metal substrate and various cold-formed products. In one or more embodiments, the cold-formed products include a structural substrate comprising a curved surface and structural substrate coefficient of thermal expansion (CTE), a cold-formed and curved glass substrate attached to the curved surface with an adhesive, the glass substrate comprising a glass substrate CTE, the structural substrate and adhesive forming a structural substrate/adhesive interface and the glass substrate and the adhesive forming a glass substrate/adhesive interface, wherein the glass substrate CTE and the structural substrate CTE differ, wherein the product withstands overlap shear failure as determined by modified test method ASTM D1002-10 at −40° C., 24° C., and 85° C. and tensile failure as determined by ASTM D897 at −40° C., 24° C., and 85° C. at one or both of the structural substrate/adhesive interface and the glass substrate/adhesive interface.

### CROSS-REFERENCE TO RELATED APPLICATIONS

This application claims the benefit of priority under 35 U.S.C. § 119 of U.S. Provisional Application Ser. No. 62/814,906 filed on Mar. 7, 2019 and U.S. Provisional Application Ser. No. 62/755,203 filed on Nov. 2, 2018 and U.S. Provisional Application Ser. No. 62/747,531 filed on Oct. 18, 2018, the content of which are relied upon and incorporated herein by reference in their entirety.

### BACKGROUND

Automotive interiors can include curved surfaces that incorporate displays and/or touch panel. The materials used to form such curved surfaces are typically limited to polymers, which do not exhibit the durability and optical performance of glass. As such, curved glass substrates are desirable, especially when used as covers for displays and/or touch panels. Existing methods of forming curved glass substrates, such as thermal forming, have drawbacks including high cost, and optical distortion and/or surface marking occurring during curving or shaping. Accordingly, there is a need for automotive interior systems that can incorporate a curved glass substrate in a cost-effective manner and without the problems typically associated with glass thermal forming processes. Further, there is a need for methods that allow for the rapid selection of adhesives that maintain the adequate bonding of curved glass substrates to surfaces in an automotive interior, such that the bonded curved glass substrates will have instantaneous survivability and reliability substantially over the entire life of an automotive.

### SUMMARY

A first aspect of this disclosure pertains to various methods for the selection of adhesives that maintain the adequate bonding of curved glass substrates to surfaces in an automotive interior or subcomponents of an automotive interior (e.g., a structural substrate, such as a frame). In addition, the adhesives selected by the methods disclosed herein, will lead to bonded curved glass substrates having instantaneous survivability and reliability substantially over the entire life of an automotive. For example, adhesives selected using the methods described herein will be able to withstand the stresses and strains in cold-formed curved glass substrates that are bonded with those adhesives to a structural substrate (e.g., a frame), without delamination for a period of time (e.g., 15 years or more).

One or more embodiments pertains to a cold-formed product comprising: a structural substrate comprising a curved surface of defined radius of curvature and structural substrate coefficient of thermal expansion (CTE); a cold-formed and curved glass substrate attached to the curved surface with an adhesive, the glass substrate having a glass radius of curvature and comprising a glass substrate CTE, the structural substrate and adhesive forming a structural substrate/adhesive interface and the glass substrate and the adhesive forming a glass substrate/adhesive interface, wherein the glass substrate CTE and the structural substrate CTE differ, wherein the product withstands overlap shear failure as determined by modified test method ASTM D1002-10 at −40° C., 24° C., and 85° C. and tensile failure as determined by ASTM D897 at −40° C., 24° C., and 85° C. at one or both of the structural substrate/adhesive interface and the glass substrate/adhesive interface.

### DESCRIPTION OF THE DRAWINGS

The drawings illustrate generally, by way of example, but not by way of limitation, various embodiments discussed herein.

FIG. 1 is a perspective view illustration of an automotive interior with automotive interior systems according to one or more embodiments.

FIG. 2 is a side view illustration of a display including a curved glass substrate with no flat tip.

FIG. 3 is a side view illustration of the glass substrate used in the display of FIG. 2.

FIG. 4 is a front perspective view illustration of the glass substrate of FIG. 3.

FIG. 5 is a diagram of an example system 500.

FIG. 6 is a flow chart for an example method 600.

FIG. 7 is a is a block diagram illustrating components of a machine 1600.

FIG. 8 is a diagram of a modified ASTM D897 stack.

FIGS. 9 and 10 are plots of adhesive modulus as a function of substrate (e.g., a frame) CTE for radius greater than 400 mm (part length greater than or equal to 200 mm) and radius of greater than 150 mm but less than or equal to 400 mm (part length greater than or equal to 200 mm).

Repeated use of reference characters in the specification and drawings is intended to represent the same or analogous features or elements of the disclosure, even when the numbers increase by 100 from figure-to-figure. It should be understood that numerous other modifications and examples can be devised by those skilled in the art, which fall within the scope and spirit of the principles of the disclosure.

### DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Cold forming (e.g., bending) is an energy efficient method of creating curved glass substrates based on the elastic deformation of glass at relatively low temperature (e.g., <140° C.) with the application of out of plane loads to create the desired shape. During the cold forming process, a flat high-strength glass is three-dimensionally (3D) deformed and mechanically fixed by an adhesive interlayer to a target pre-formed 3D frame to which, e.g., display functional modules are mounted. This cold forming process results in stresses in the resulting curved glass substrates, the adhesive layer, and the target frame.

The produced mechanical stresses in the adhesive due to glass bending will last throughout its lifetime. The mechanical stresses can cause not only instantaneous failure of the adhesive, but also long-term reliability issues. Desired stress thresholds of adhesives are some of the critical values to determine its instantaneous survivability and long-term reliability. The thresholds are varied depending on adhesive types, temperature conditions, glass mechanical properties, material type, and geometry of the pre-formed 3D frame.

Existing methods that can reduce adhesive stresses to meet requirement of instantaneous survivability and long-term reliability includes the concept of flat-tip or flat portion of glass. A first aspect of this disclosure provides methods for the selection of adhesive families, as well as specific adhesives, with or without the glass flat tip, for cold forming of curved glass substrates. The adhesive families, and specific adhesives selected using the methods described herein meet requirement of instantaneous survivability and long-term reliability, based on adhesive type and its mechanical and thermal properties; adhesive thickness; frame material type and its mechanical and thermal properties; frame thickness; and glass thickness.

In one or more embodiments, the method for selecting an adhesive for forming a cold-formed product (as described herein according to various embodiments) comprising: calculating at least one of the ambient stress and ambient strain of the adhesive on a substrate; calculating an ambient stress to strength ratio of the adhesive;

calculating at least one of the stress and strain of the adhesive on the substrate as a function of temperature; and calculating a stress to strength ratio of the adhesive as a function of temperature; and
selecting the adhesive if the ambient stress to strength ratio changes less than an order of magnitude as a function of time.

As used herein, the term “strength” refers to the tensile strength or the shear strength.

In one or more embodiments, the resulting cold-formed product may include a combination of more than one adhesive to bond a structural substrate to a glass. For example, one can select one adhesive for one part of the structural substrate, and another one for a different part of the structural substrate. In so doing, one can, e.g., select a softer adhesive in areas of low stress and stronger adhesive in area of high stress.

In one or more embodiments of the method, the ambient stress to strength ratio as a function of time can change for example, from about 3:10 to about 3:100 as a function of time. For example, the ambient stress to strength ratio as a function of time can change from about 3:10 to about 3:50, about 3:10 to about 3:75, about 3:10 to about 1:10, about 1:10 to about 1:100, about 1:100 to about 3:100, about 3:10 to about 1:100, about 2:10 to about 1:50 or about 3:50 to about 3:90, for example, as a function of a time period of at least about 5 years, at least about 10 years, at least about 15 years or longer; or over a 15 year time period.

The cold-formed glass, metal substrate, and the adhesive that bonds the cold-formed glass to the metal substrate can be found in an automotive interior system. The automotive interior system, in turn, can be incorporated into any automotive, including trains, automobiles (e.g., cars, trucks, buses and the like), seacraft (boats, ships, submarines, and the like), and aircraft (e.g., drones, airplanes, jets, helicopters and the like).

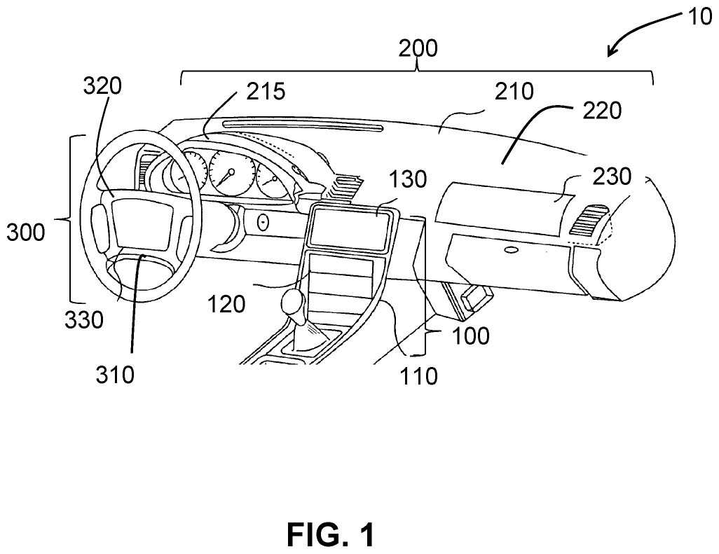

FIG. 1 provides an example of an automotive interior 10, including automotive interior systems 100, 200, 300. Automotive interior system 100 includes a center console base 110 with a curved surface 120 including a display 130. Automotive interior system 200 includes a dashboard base 210 with a curved surface 220 including a display 230. The dashboard base 210 typically includes an instrument panel 215 which may also include a display. Automotive interior system 300 includes a dashboard steering wheel base 310 with a curved surface 320 and a display 330. In one or more embodiments, the automotive interior system may include a base that is an arm rest, a pillar, a seat back, a floor board, a headrest, a door panel, or any portion of the interior of an automotive that includes a curved surface.

The cold-formed glass substrates described herein can be used as curved cover glasses for any of the display embodiments described herein, including for use in automotive interior systems 100, 200 and/or 300. As used herein, the term “glass substrate” is used in its broadest sense to include any object made wholly or partly of glass. Glass substrates include laminates of glass and non-glass materials, laminates of glass and crystalline materials, and glass-ceramics (including an amorphous phase and a crystalline phase). The glass substrate may be transparent or opaque. Cold-formed glass substrate can include a colorant that provides a specific color. Suitable glass compositions for use in the cold-formed glass substrates described herein include soda lime glass, aluminosilicate glass, borosilicate glass, boroaluminosilicate glass, alkali-containing aluminosilicate glass, alkali-containing borosilicate glass, and alkali-containing boroaluminosilicate glass. The glass substrate may be optionally strengthened. For example, the glass substrate can be strengthened using any suitable method known in the art and may exhibit a compressive stress (CS) that extends from a surface to a depth of compression (DOC). In one or more embodiments, the strengthened glass substrate may be a mechanically strengthened glass substrate in which CS is generated by utilizing a mismatch of the coefficient of thermal expansion between portions of the glass substrate to create a compressive stress region at the opposing surface portions and a central region exhibiting a tensile stress. In one or more embodiments, In one or more embodiments, the strengthened glass substrate may be a mechanically strengthened glass substrate in which CS is generated thermally by heating the glass substrate to a temperature above the glass transition point and then rapidly quenching. In one or more embodiments, In one or more embodiments, the strengthened glass substrate may be a mechanically strengthened glass substrate in which CS is generated chemically by ion exchange, where, e.g., ions at or near the surface of the glass substrate are replaced by, or exchanged with, larger ions having the same valence or oxidation state.

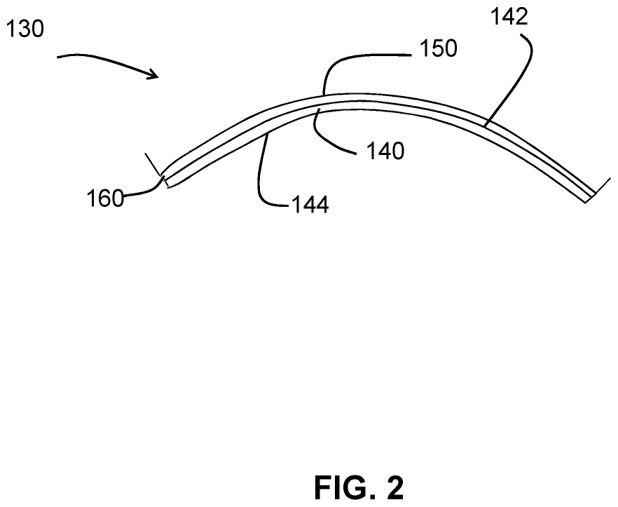

As shown in FIG. 2, the display 130 includes cold-formed curved glass substrate 140 having a first radius of curvature and a frame 150 (e.g., a metal frame, made of stainless steel or aluminum), and an adhesive layer 160 located between the glass substrate 140 and the frame 150, wherein at least a portion of the frame 150 has a second radius of curvature that approximates or matches the first radius of curvature, to provide a display 130 with a curved glass substrate as a cover glass that can be integrated into a curved surface of an automotive interior system. Convex or concave displays, as well as displays having both convex and concave features, are contemplated herein.

The cold-formed curved glass substrate shown in FIG. 2 does not have a flat tip. But those of skill in the art will recognize that the cold-formed curved glass substrate shown in FIG. 2 can have a flat tip that can range in width (w_{ft}) from about 30 mm to about 100 mm.

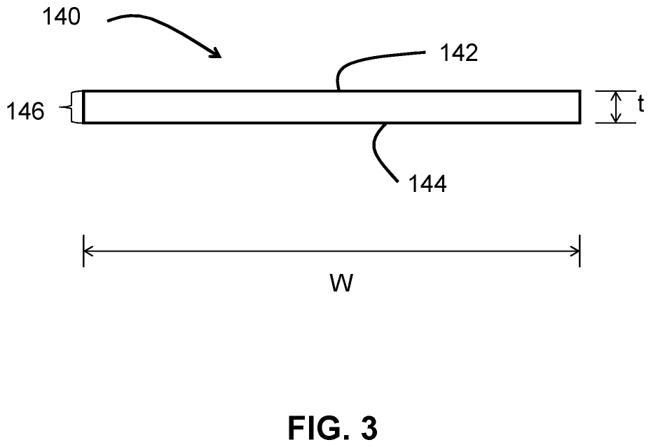

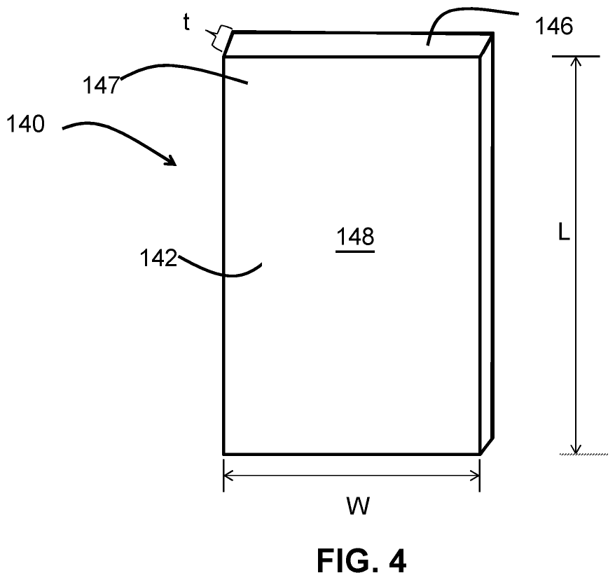

Making reference to FIGS. 3 and 4, the cold-formed glass substrate 140 includes a first major surface 142 and a second major surface 144 opposite the first major surface. The cold-formed glass substrate exhibits the first radius of curvature as measured on the second major surface 144.

As used herein, the terms “cold-formed,” “cold-bent,” or “cold-bending” refers to curving the glass substrate at a cold-forming temperature which is less than the softening point of the glass. The term “cold-bendable” refers to the capability of a glass substrate to be cold-bent. A feature of a cold-formed glass substrate is asymmetric surface compressive stress between the first major surface 142 and the second major surface 144. A minor surface 146 connects the first major surface 142 and the second major surface 144. Prior to the cold-forming process or being cold-formed, the respective compressive stresses in the first major surface 142 and the second major surface 144 of the glass substrate are substantially equal. When the glass substrate is unstrengthened, the first major surface 142 and the second major surface 144 exhibit no appreciable compressive stress, prior to cold-forming. When the glass substrate is strengthened, the first major surface 142 and the second major surface 144 exhibit substantially equal compressive stress with respect to one another, prior to cold-forming.

After cold-forming (shown, for example, in FIG. 2), the compressive stress on the surface having a concave shape after bending (e.g., first major surface 142 in FIG. 2) increases. In other words, the compressive stress on the concave surface (e.g., first major surface 142) is greater after cold-forming than before cold-bending. Without being bound by theory, the cold-bending process increases the compressive stress of the glass substrate being shaped to compensate for tensile stresses imparted during bending and/or forming operations. The cold-forming process causes the concave surface (second major surface 144) to experience compressive stresses, while the surface forming a convex shape (e.g., the second major surface 144 in FIG. 2) after cold-forming experiences tensile stresses. The tensile stress experienced by the convex (e.g., the second major surface 144) following cold-forming results in a net decrease in surface compressive stress, such that the compressive stress in convex surface (e.g., the second major surface 144) of a strengthened glass substrate following cold-forming is less than the compressive stress on the same surface (e.g., second major surface 144) when the glass substrate is flat.

When a strengthened glass substrate is utilized, the first major surface and the second major surface (142, 144) comprise a compressive stress that is substantially equal to one another prior to cold-forming, and thus the first major surface can experience greater tensile stress during cold-bending without risking fracture. This allows for the strengthened glass substrate to conform to more tightly curved surfaces or shapes.

The thickness of the glass substrate can be tailored to allow the glass substrate to be more flexible to achieve the desired radius of curvature. Moreover, a thinner glass substrate 140 may deform more readily, which could potentially compensate for shape mismatches and gaps that may be created by the shape of the display module 150 (when curved). In one or more embodiments, a thin and strengthened glass substrate 140 exhibits greater flexibility especially during cold-bending. The greater flexibility of the glass substrates discussed herein may both allow for sufficient degrees of bending to be created via the air pressure-based bending processes as discussed herein and also for consistent bend formation without heating. The glass substrate 140 and at least a portion of the display module 150 have substantially similar radii of curvature to provide a substantially uniform distance between the first major surface 142 and the display module 150, which can be filled with an adhesive.

The cold-formed glass substrate (and optionally the curved display module) can have a compound curve including a major radius and a cross curvature. A complexly curved cold-formed glass substrate (and optionally the curved display module) can have a distinct radius of curvature in two independent directions. The complexly curved cold-formed glass substrate (and optionally the curved display module) can thus be characterized as having “cross curvature,” where the cold-formed glass substrate (and optionally the curved display module) are curved along an axis (e.g., a first axis) that is parallel to a given dimension and also curved along an axis (e.g., a second axis) that is perpendicular to the same dimension. The curvature of the cold-formed glass substrate (and optionally the curved display module) can be even more complex when a significant minimum radius is combined with a significant cross curvature, and/or depth of bend.

The cold-formed glass substrate has a thickness (t) that is substantially constant and is defined as a distance between the first major surface 142 and the second major surface 144. The thickness (t) as used herein refers to the maximum thickness of the glass substrate. As shown in FIGS. 3-4, the glass substrate includes a width (W) defined as a first maximum dimension of one of the first or second major surfaces orthogonal to the thickness (t), and a length (L) defined as a second maximum dimension of one of the first or second surfaces orthogonal to both the thickness and the width. The dimensions discussed herein can be average dimensions.

As used herein, the term “ambient stress and ambient strain” generally refers to the stress and strain that a material is exposed to at a temperature of about 20-25° C. and a pressure of 101325 Pa.

The calculating of at least one of the ambient stress and ambient strain of the adhesive on the metal substrate can be based on at least one of a thickness of the cold-formed glass, a thickness of the metal substrate, and a thickness of the adhesive. Alternatively, or in addition, the calculating of at least one of the ambient stress and ambient strain of the adhesive on the metal substrate is based on a physical property of at least one of the cold-formed glass, the metal substrate, and the adhesive. Physical properties of the at least one of the cold-formed glass, the metal substrate, and the adhesive include, but are not limited to the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature (T_{g}) of the adhesive.

The Young's modulus of a material is a measure of the linear elastic response of the material, i.e., when a loaded material is unloaded, it returns to its original undeformed state and is typically observed at very low strains of about less than 5% or less than 3% or from about 1% to about 3%. Viscoelastic materials exhibit both elastic and viscous characteristics, as well as show hysteresis in loading-unloading curve. Hyperelastic material models can be either phenomenological or mechanistic model or hybrid.

Those of skill in the art will recognize that the T_{g} of a material depends strongly on the cure schedule (e.g., temperature and time duration at that temperature) at which the material is cured (e.g., crosslinked). Hence, the same adhesive cured at different temperature/time schedule could have different T_{g} depending on its crosslink density.

The calculating of at least one of the ambient stress and ambient strain of the adhesive on the metal substrate can be based on a bending radius, or radius of curvature, of a cold-formed glass substrate. The radius of curvature can be, for example, about 20 mm or greater, 40 mm or greater, 50 mm or greater, 60 mm or greater, 100 mm or greater, 250 mm or greater or 500 mm or greater. For example, the first radius of curvature may be in a range from about 20 mm to about 10000 mm, from about 30 mm to about 10000 mm, from about 40 mm to about 1500 mm, from about 50 mm to about 1500 mm, 60 mm to about 1500 mm, from about 70 mm to about 10000 mm, from about 80 mm to about 1500 mm, from about 90 mm to about 10000 mm, from about 100 mm to about 10000 mm, from about 120 mm to about 10000 mm, from about 140 mm to about 10000 mm, from about 150 mm to about 10000 mm, from about 160 mm to about 10000 mm, from about 180 mm to about 10000 mm, from about 200 mm to about 10000 mm, from about 220 mm to about 10000 mm, from about 240 mm to about 10000 mm, from about 250 mm to about 10000 mm, from about 260 mm to about 10000 mm, from about 270 mm to about 10000 mm, from about 280 mm to about 10000 mm, from about 290 mm to about 10000 mm, from about 300 mm to about 10000 mm, from about 350 mm to about 10000 mm, from about 400 mm to about 10000 mm, from about 450 mm to about 10000 mm, from about 500 mm to about 10000 mm, from about 550 mm to about 10000 mm, from about 600 mm to about 10000 mm, from about 650 mm to about 10000 mm, from about 700 mm to about 10000 mm, from about 750 mm to about 10000 mm, from about 800 mm to about 10000 mm, from about 900 mm to about 10000 mm, from about 950 mm to about 10000 mm, from about 1000 mm to about 10000 mm, from about 1250 mm to about 10000 mm, from about 1500 mm to about 10000 mm, from about 1750 mm to about 10000 mm, from about 2000 mm to about 10000 mm, from about 3000 mm to about 10000 mm, from about 20 mm to about 9000 mm, from about 20 mm to about 8000 mm, from about 20 mm to about 7000 mm, from about 20 mm to about 6000 mm, from about 20 mm to about 5000 mm, from about 20 mm to about 4000 mm, from about 20 mm to about 3000 mm, from about 20 mm to about 2500 mm, from about 20 mm to about 2000 mm, from about 20 mm to about 1750 mm, from about 20 mm to about 1500 mm, from about 20 mm to about 1400 mm, from about 20 mm to about 1300 mm, from about 20 mm to about 1200 mm, from about 20 mm to about 1100 mm, from about 20 mm to about 1000 mm, from about 20 mm to about 950 mm, from about 20 mm to about 900 mm, from about 20 mm to about 850 mm, from about 20 mm to about 800 mm, from about 20 mm to about 750 mm, from about 20 mm to about 700 mm, from about 20 mm to about 650 mm, from about 20 mm to about 200 mm, from about 20 mm to about 550 mm, from about 20 mm to about 500 mm, from about 20 mm to about 450 mm, from about 20 mm to about 400 mm, from about 20 mm to about 350 mm, from about 20 mm to about 300 mm, from about 20 mm to about 250 mm, from about 20 mm to about 200 mm, from about 20 mm to about 150 mm, from about 20 mm to about 100 mm, from about 20 mm to about 50 mm, from about 60 mm to about 1400 mm, from about 60 mm to about 1300 mm, from about 60 mm to about 1200 mm, from about 60 mm to about 1100 mm, from about 60 mm to about 1000 mm, from about 60 mm to about 950 mm, from about 60 mm to about 900 mm, from about 60 mm to about 850 mm, from about 60 mm to about 800 mm, from about 60 mm to about 750 mm, from about 60 mm to about 700 mm, from about 60 mm to about 650 mm, from about 60 mm to about 600 mm, from about 60 mm to about 550 mm, from about 60 mm to about 500 mm, from about 60 mm to about 450 mm, from about 60 mm to about 400 mm, from about 60 mm to about 350 mm, from about 60 mm to about 300 mm, or from about 60 mm to about 250 mm. In one or more embodiments, glass substrates having a thickness of less than about 0.4 mm may exhibit a radius of curvature that is less than about 100 mm, or less than about 60 mm.

The calculating at least one of the stress and strain of the adhesive on the metal substrate as a function of temperature is based on at least one of a thickness of the cold-formed glass, a thickness of the metal substrate, and a thickness of the adhesive.

The cold-formed glass substrate can have any suitable thickness. For example, can have a thickness (t) that is about 1.5 mm or less. For example, the thickness may be in a range from about 0.01 mm to about 1.5 mm, 0.02 mm to about 1.5 mm, 0.03 mm to about 1.5 mm, 0.04 mm to about 1.5 mm, 0.05 mm to about 1.5 mm, 0.06 mm to about 1.5 mm, 0.07 mm to about 1.5 mm, 0.08 mm to about 1.5 mm, 0.09 mm to about 1.5 mm, 0.1 mm to about 1.5 mm, from about 0.15 mm to about 1.5 mm, from about 0.2 mm to about 1.5 mm, from about 0.25 mm to about 1.5 mm, from about 0.3 mm to about 1.5 mm, from about 0.35 mm to about 1.5 mm, from about 0.4 mm to about 1.5 mm, from about 0.45 mm to about 1.5 mm, from about 0.5 mm to about 1.5 mm, from about 0.55 mm to about 1.5 mm, from about 0.6 mm to about 1.5 mm, from about 0.65 mm to about 1.5 mm, from about 0.7 mm to about 1.5 mm, from about 0.01 mm to about 1.4 mm, from about 0.01 mm to about 1.3 mm, from about 0.01 mm to about 1.2 mm, from about 0.01 mm to about 1.1 mm, from about 0.01 mm to about 1.05 mm, from about 0.01 mm to about 1 mm, from about 0.01 mm to about 0.95 mm, from about 0.01 mm to about 0.9 mm, from about 0.01 mm to about 0.85 mm, from about 0.01 mm to about 0.8 mm, from about 0.01 mm to about 0.75 mm, from about 0.01 mm to about 0.7 mm, from about 0.01 mm to about 0.65 mm, from about 0.01 mm to about 0.6 mm, from about 0.01 mm to about 0.55 mm, from about 0.01 mm to about 0.5 mm, from about 0.01 mm to about 0.4 mm, from about 0.01 mm to about 0.3 mm, from about 0.01 mm to about 0.2 mm, from about 0.01 mm to about 0.1 mm, from about 0.04 mm to about 0.07 mm, from about 0.1 mm to about 1.4 mm, from about 0.1 mm to about 1.3 mm, from about 0.1 mm to about 1.2 mm, from about 0.1 mm to about 1.1 mm, from about 0.1 mm to about 1.05 mm, from about 0.1 mm to about 1 mm, from about 0.1 mm to about 0.95 mm, from about 0.1 mm to about 0.9 mm, from about 0.1 mm to about 0.85 mm, from about 0.1 mm to about 0.8 mm, from about 0.1 mm to about 0.75 mm, from about 0.1 mm to about 0.7 mm, from about 0.1 mm to about 0.65 mm, from about 0.1 mm to about 0.6 mm, from about 0.1 mm to about 0.55 mm, from about 0.1 mm to about 0.5 mm, from about 0.1 mm to about 0.4 mm, or from about 0.3 mm to about 0.7 mm.

The cold-formed glass substrate can also have a width (W) in a range from about 5 cm to about 250 cm, from about 5 cm to about 20 cm, from about 10 cm to about 250 cm, from about 15 cm to about 250 cm, from about 20 cm to about 250 cm, from about 25 cm to about 250 cm, from about 30 cm to about 250 cm, from about 35 cm to about 250 cm, from about 40 cm to about 250 cm, from about 45 cm to about 250 cm, from about 50 cm to about 250 cm, from about 55 cm to about 250 cm, from about 60 cm to about 250 cm, from about 65 cm to about 250 cm, from about 70 cm to about 250 cm, from about 75 cm to about 250 cm, from about 80 cm to about 250 cm, from about 85 cm to about 250 cm, from about 90 cm to about 250 cm, from about 95 cm to about 250 cm, from about 100 cm to about 250 cm, from about 110 cm to about 250 cm, from about 120 cm to about 250 cm, from about 130 cm to about 250 cm, from about 140 cm to about 250 cm, from about 150 cm to about 250 cm, from about 5 cm to about 240 cm, from about 5 cm to about 230 cm, from about 5 cm to about 220 cm, from about 5 cm to about 210 cm, from about 5 cm to about 200 cm, from about 5 cm to about 190 cm, from about 5 cm to about 180 cm, from about 5 cm to about 170 cm, from about 5 cm to about 160 cm, from about 5 cm to about 150 cm, from about 5 cm to about 140 cm, from about 5 cm to about 130 cm, from about 5 cm to about 120 cm, from about 5 cm to about 110 cm, from about 5 cm to about 110 cm, from about 5 cm to about 100 cm, from about 5 cm to about 90 cm, from about 5 cm to about 80 cm, or from about 5 cm to about 75 cm.

The cold-formed glass substrate can also have a length (L) in a range from about 5 cm to about 250 cm, from about 30 cm to about 90 cm, from about 10 cm to about 250 cm, from about 15 cm to about 250 cm, from about 20 cm to about 250 cm, from about 25 cm to about 250 cm, from about 30 cm to about 250 cm, from about 35 cm to about 250 cm, from about 40 cm to about 250 cm, from about 45 cm to about 250 cm, from about 50 cm to about 250 cm, from about 55 cm to about 250 cm, from about 60 cm to about 250 cm, from about 65 cm to about 250 cm, from about 70 cm to about 250 cm, from about 75 cm to about 250 cm, from about 80 cm to about 250 cm, from about 85 cm to about 250 cm, from about 90 cm to about 250 cm, from about 95 cm to about 250 cm, from about 100 cm to about 250 cm, from about 110 cm to about 250 cm, from about 120 cm to about 250 cm, from about 130 cm to about 250 cm, from about 140 cm to about 250 cm, from about 150 cm to about 250 cm, from about 5 cm to about 240 cm, from about 5 cm to about 230 cm, from about 5 cm to about 220 cm, from about 5 cm to about 210 cm, from about 5 cm to about 200 cm, from about 5 cm to about 190 cm, from about 5 cm to about 180 cm, from about 5 cm to about 170 cm, from about 5 cm to about 160 cm, from about 5 cm to about 150 cm, from about 5 cm to about 140 cm, from about 5 cm to about 130 cm, from about 5 cm to about 120 cm, from about 5 cm to about 110 cm, from about 5 cm to about 110 cm, from about 5 cm to about 100 cm, from about 5 cm to about 90 cm, from about 5 cm to about 80 cm, or from about 5 cm to about 75 cm.

The structural substrate can have any suitable thickness. For example, the structural substrate thickness can be in a range from about 0.5 mm to about 20 mm (e.g., from about 2 mm to about 20 mm, from about 3 mm to about 20 mm, from about 4 mm to about 20 mm, from about 5 mm to about 20 mm, from about 6 mm to about 20 mm, from about 7 mm to about 20 mm, from about 8 mm to about 20 mm, from about 9 mm to about 20 mm, from about 10 mm to about 20 mm, from about 12 mm to about 20 mm, from about 14 mm to about 20 mm, from about 1 mm to about 18 mm, from about 1 mm to about 16 mm, from about 1 mm to about 15 mm, from about 1 mm to about 14 mm, from about 1 mm to about 12 mm, from about 1 mm to about 10 mm, from about 1 mm to about 8 mm, from about 1 mm to about 6 mm, from about 1 mm to about 5 mm, from about 1 mm to about 4 mm, from about 1 mm to about 3 mm, from about 1 mm to about 2 mm, and all ranges and sub-ranges therebetween.

The adhesive can have any suitable thickness, measured from a surface of the adhesive that contacts the cold-formed glass substrate to a surface of the metal substrate, as shown in FIG. 1. The thickness of the adhesive can be tailored to, among other things, ensure lamination between the metal substrate and the cold-formed glass substrate. For example, the adhesive may have a thickness of about 5 mm or less, about 4 mm or less, about 3 mm or less, 2.5 mm or less, about 2 mm or less, about 1.5 mm or less, or about 1 mm or less. The adhesive can have a thickness in a range from about 200 μm to about 2 mm μm, about 200 μm to about 1.75 mm, about 200 μm to about 1.5 mm, about 200 μm to about 1.25 mm, about 200 μm to about 1 mm, about 200 μm to about 750 μm, about 200 μm to about 500 μm, from about 225 μm to about 500 μm, from about 250 μm to about 500 μm, from about 275 μm to about 500 μm, from about 300 μm to about 500 μm, from about 325 μm to about 500 μm, from about 350 μm to about 500 μm, from about 375 μm to about 500 μm, from about 400 μm to about 500 μm, from about 200 μm to about 475 μm, from about 200 μm to about 450 μm, from about 200 μm to about 425 μm, from about 200 μm to about 400 μm, from about 200 μm to about 375 μm, from about 200 μm to about 350 μm, from about 200 μm to about 325 μm, from about 200 μm to about 300 μm, or from about 225 μm to about 275 μm.

The adhesive can also have any suitable bezel width. For example, can have a bezel width of about 25 mm or less. The adhesive can have a bezel width in a range from about 1 mm to about 15 mm, from about 5 mm to about 20 mm, from about 10 mm to about 15 mm, from about 1 mm to about 10 mm, from about 5 mm to about 10 mm, from about 5 mm to about 15 mm, from about 10 mm to about 20 mm, or from about 1 mm to about 5 mm.

The methods described herein involve a step of calculating at least one of the stress and strain of the adhesive on the metal substrate as a function of temperature. The calculating at least one of the stress and strain of the adhesive on the metal substrate as a function of temperature can be based on a physical property of at least one of the cold-formed glass, the metal substrate, and the adhesive, such as, the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature of the adhesive. The calculating at least one of the stress and strain of the adhesive on the metal substrate as a function of temperature can be based on the bending radius of the cold-formed glass.

Suitable adhesive used in the cold-formed product or adhesives selected using the methods described herein can be an intermediate adhesive. Examples of adhesives that can be selected using the methods or used in the cold-formed product described herein include polyurethanes (e.g., DP604NS available from 3M®, Saint Paul, Minn., as well as Betamate 73100/002, 73100/005, 73100/010, Betaseal X2500, and Betalink K2, from Dupont®, Wilmington, Del.), polysiloxanes and silane-modified polymers (e.g., TEROSON RB IX, also known as TEROSTAT MS 9399 and TEROSON MS 647, available from Loctite®), and epoxies (e.g., Scotch-Weld™ Epoxy Adhesive DP125 and DP604 available from 3M®, Saint Paul, Minn.). Additional adhesives include, but not limited to, an adhesive selected from one of more of the categories: (a) Toughened Epoxy (for example, Masterbond EP21TDCHT-LO, 3M Scotch Weld Epoxy DP460 Off-white); (b) Flexible Epoxy (for example, Masterbond EP21TDC-2LO, 3M Scotch Weld Epoxy 2216); (c) Acrylics and/or Toughened Acrylics (for example, LORD Adhesive 403, 406 or 410 Acrylic adhesives with LORD Accelerator 19 or 19 GB w/LORD AP 134 primer, LORD Adhesive 850 or 852/LORD Accelerator 25 GB, Loctite HF8000, Loctite AA4800); (d) Urethanes (for example, 3M Scotch Weld Urethane DP640 Brown, SikaForce 7570 L03, SikaForce 7550 L15, Sikaflex 552 and Polyurethane (PUR) Hot Melt adhesives such as, Technomelt PUR 9622-02 UVNA, Loctite HHD 3542, Loctite HHD 3580, 3M Hotmelt adhesives 3764 and 3748); and (e) Silicones (Dow Corning 995, Dow Corning 3-0500 Silicone Assembly adhesive, Dow Corning 7091, SikaSil-GP). In some cases, structural adhesives available as sheets or films (for example, but not limited to, 3M Structural adhesive films AF126-2, AF 163-2M, SBT 9263 and 9214, Masterbond FLM36-LO) may be utilized. Furthermore, pressure sensitive adhesives such as 3M VHB tapes may be utilized. In such embodiments, utilizing a pressure sensitive adhesive allows for the curved glass substrate to be bonded to the frame without the need for, among other things, a curing step.

TABLE 1
Examples of tensile strength and overlap shear strength of “stack” constructions,
and at the interfaces of the stack, at a temperature of −40° C., 24° C., and 85° C.

| | | Tensile Strength (MPa) | | |
|---|---|---|---|---|
| | Adhesive | −40° C. | 24° C. | 85° C. |
| Toughened | 3M DP460 Epoxy | 49.7 ± 5.3 | 23.2 ± 3.0 | 7.2 ± 1.9 |
| Epoxy | (cured at 66° C. for 1 hr.) | | | |
| | EP21TDCHT-LO | 30.3 ± 1.5 | 19.2 ± 1.3 | 9.4 ± 1.6 |
| | (cured at 66° C. for 3 hr.) | | | |
| Flexible Epoxy | Epoxy 2216 | 49.0 ± 9.5 | 20.4 ± 3.3 | 3.8 ± 0.7 |
| | (cured at 66° C. for 2 hr.) | | | |
| | EP21TDC-LO | 17.1 ± 1.8 | 4.9 ± 0.2 | 2.1 ± 0.2 |
| | (cured at 66° C. for 4 hr.) | | | |
| | DP125 | 29.2 ± 4.6 | 12.2 ± 0.6 | 3.55 ± 1.4 |
| | (cured at 66° C. for 1 hr.) | | | |
| Polyurethane | DP640 | 20.0 ± 3.2 | 12.3 ± 2.2 | 3.2 ± 0.5 |
| | (cured at 66° C. for 5 hr.) | | | |
| | DP604NS | 17.1 ± 1.3 | 8.2 ± 1.8 | 4.8 ± 0.6 |
| | (cured at 66° C. for 20 min.) | | | |
| Acrylic | LORD850/ACC. | 15.8 ± 3.6 | 8.7 ± 1.0 | 3.0 ± 1.3 |
| | 24GB/AP-134 | | | |
| | (cured at 24° C. for 2 hr.) | | | |
| Silicone | Dow Corning 7091 | 1.39 ± 0.11 | 1.08 ± 0.12 | 0.98 ± 0.13 |
| | (cured at 24° C. for 48 hr.) | | | |
| | Teroson MS 9399 | 4.6 ± 1.7 | 2.0 ± 0.02 | 1.42 ± 0.14 |
| | (cured at 24° C. for 48 hr.) | | | |
| Acrylic foam | 3M VHB tape 5952 | 1.0 ± 0.1 | 0.24 ± 0.03 | 0.12 ± 0.01 |
| tape | (cured at 24° C. for 48 hr.) | | | |
| | 3M VHB tape 4611 | 3.63 ± 1.23 | 0.26 ± 0.02 | 0.11 ± 0.01 |
| | (cured at 24° C. for 48 hr.) | | | |
| | | Overlap Shear Strength (MPa) | | |
| | Adhesive | −40° C. | 24° C. | 85° C. |
| Toughened | 3M DP460 Epoxy | 14.5 ± 3.6 | 15.6 ± 5.0 | 5.0 ± 1.7 |
| Epoxy | (cured at 66° C. for 1 hr.) | | | |
| | EP21TDCHT-LO | — | — | — |
| | (cured at 66° C. for 3 hr.) | | | |
| Flexible Epoxy | Epoxy 2216 | 17.5 ± 2.9 | 10.6 ± 0.2 | — |
| | (cured at 66° C. for 2 hr.) | | | |
| | EP21TDC-LO | 17.1 ± 1.8 | 4.9 ± 0.2 | 2.1 ± 0.2 |
| | (cured at 66° C. for 4 hr.) | | | |
| | DP125 | 13.5 ± 3.6 | 7.5 ± 0.8 | 0.9 ± 0.2 |
| | (cured at 66° C. for 1 hr.) | | | |
| Polyurethane | DP640 | 7.2 ± 1.0 | 3.5 ± 1.5 | 0.9 ± 0.1 |
| | (cured at 66° C. for 5 hr.) | | | |
| | DP604NS | 6.1 ± 1.8 | 3.0 ± 0.9 | 2.7 ± 0.3 |
| | (cured at 66° C. for 20 min.) | | | |
| Acrylic | LORD850/ACC. | — | — | — |
| | 24GB/AP-134 | | | |
| | (cured at 24° C. for 2 hr.) | | | |
| Silicone | Dow Corning 7091 | 1.4 ± 0.6 | 1.4 ± 0.1 | 0.98 ± 0.2 |
| | (cured at 24° C. for 48 hr.) | | | |
| | Teroson MS 9399 | — | 1.6 ± 0.1 | 0.94 ± 0.1 |
| | (cured at 24° C. for 48 hr.) | | | |
| Acrylic foam | 3M VHB tape 5952 | 1.24 ± 0.01 | 0.21 ± 0.01 | 0.06 ± 0.0 |
| tape | (cured at 24° C. for 48 hr.) | | | |
| | 3M VHB tape 4611 | 4.49 ± 1.1 | 0.26 ± 0.05 | 0.1 ± 0.01 |
| | (cured at 24° C. for 48 hr.) | | | |

* This table was generated through a modified ASTM D897 for tensile strength and modified ASTM D10002-10 for adhesive bonding in a laminate, flat “stack,” where the “stack” comprises a decorated glass layer having a first major surface and a second major surface, where the first major surface is bonded to a first major surface of a metal substrate via an adhesive. In some instances, the decorated glass layer surface and/or the metal surface can be primed with a primer layer.

The adhesive material may be applied in a variety of ways. In one embodiment, the adhesive is applied using an applicator gun and mixing nozzle or premixed syringes or robotic adhesive dispenser, and spread uniformly using any of the following, for example, a roller, a brush, a doctor blade or a draw down bar.

The disclosure also relates to an automotive part comprising: a structural substrate having a first major surface; a cold-formed glass having a first major surface; and an adhesive having a first major surface and a second major surface; wherein: the adhesive is located between the metal substrate first major surface and the cold-formed glass first major surface; and the adhesive bonds the metal substrate first major surface to the cold-formed glass first major surface. In one or more embodiments, the adhesive may be selected using the methods described herein. The adhesive can be a polyurethane in some embodiments. In one or more embodiments, the structural substrate is a metal (e.g., steel, aluminum, plastic or a combination thereof).

The disclosure also relates to a cold-formed product comprising: a structural substrate (e.g., a frame) comprising a curved surface of defined radius of curvature and substrate coefficient of thermal expansion (CTE); a cold-formed and curved glass substrate attached to the curved surface with an adhesive, the glass substrate having a glass radius of curvature and comprising a glass substrate CTE. In one or more embodiments, the structural substrate and adhesive form a substrate/adhesive interface and the glass substrate and the adhesive forming a glass substrate/adhesive interface. In one or more embodiments, the glass CTE and the substrate CTE differ (e.g., by at least about 1%, 2%, 5%, 10%, 15% or 20%). In one or more embodiments, the cold-formed product withstands overlap shear failure as determined by modified test method ASTM D1002-10 at −40° C., 24° C., and 85° C. and tensile failure as determined by ASTM D897 at −40° C., 24° C., and 85° C. at one or both of the substrate/adhesive interface and the glass substrate/adhesive interface.

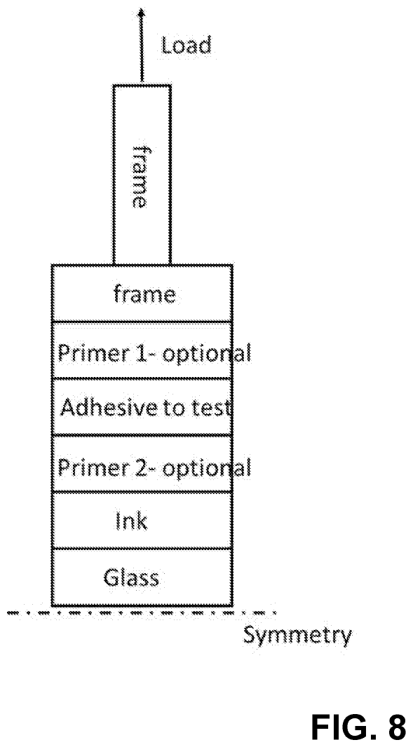

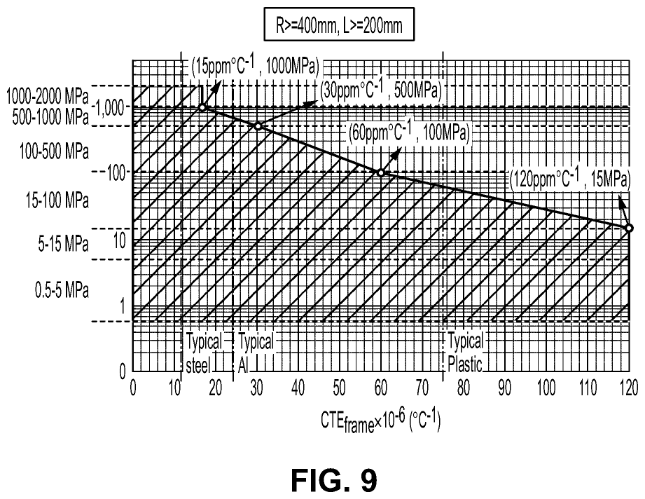

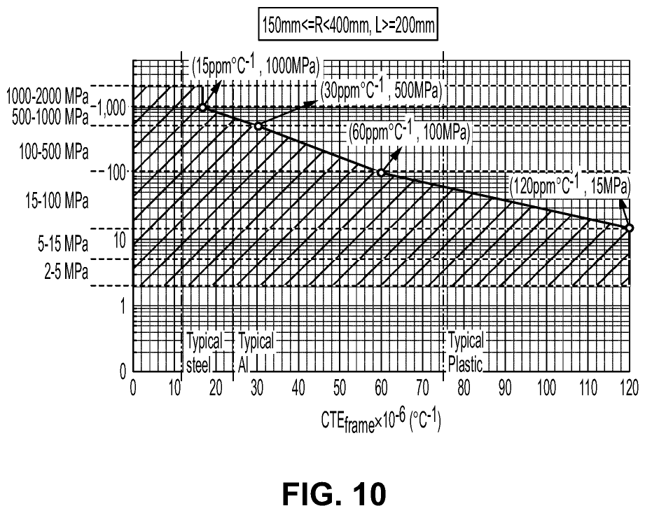

In one or more embodiments, the structural support comprises an optional primer layer (primer 1) forming a primer surface that is direct contact with the adhesive at the structural support/adhesive interface, as shown in FIG. 8. In one or more embodiments, the glass substrate has an ink layer forming an inked surface that is direct contact with the adhesive at the glass substrate/adhesive interface, as shown in FIG. 8. The cold-formed product may include an optional primer (primer 2) that is disposed between the ink layer and the adhesive and is in direct contact with the adhesive layer, as shown in FIG. 8. In any one of such embodiments, the cold-formed product withstands overlap shear failure as determined by modified test method ASTM D1002-10 at −40° C., 24° C., and 85° C. and tensile failure as determined by ASTM D897 at −40° C., 24° C., and 85° C. at one or both of the substrate/adhesive interface and the glass substrate/adhesive interface. In particular, in one or more embodiments, the cold-formed product withstands failure at the structural substrate/primer1 interface, primer1/adhesive interface, structural substrate/adhesive interface (not shown), adhesive/primer2 interface, primer2/ink layer interface, adhesive/ink interface (not shown) and ink/glass substrate interface as measured by ASTM D897 at −40° C., 24° C., and 85° C. In one or more embodiments, the cold-formed product also withstands bulk (cohesive) failure of adhesive as measured by ASTM D897 at −40° C., 24° C., and 85° C. See FIG. 8 for the arrangement of materials in the modified ASTM D897 stack. An unmodified ASTM D897 stack includes substrate-adhesive to test-substrate material. The arrangement of materials in the modified ASTM D897 stack, on the other hand, includes substrate material (e.g., frame)-primer1 (optional)-adhesive to test-primer2 (optional)-ink-glass-ink-primer (optional)-adhesive to test-primer (optional)-frame material, as shown in FIG. 8. In one or more embodiments, the radius of curvature of the cold-formed and curved glass substrate and the structural substrate can be within 10% or less of one another.

In some instances, when the glass radius of curvature is greater than or equal to about 400 mm, wherein the product comprises at least one of: an adhesive comprising a modulus in a range from about 0.5 MPa to about 5 MPa and a substrate CTE in a range from about 0 ppm/° C. to about 120 ppm/° C.; an adhesive comprising a modulus in a range from about 5 MPa to about 15 MPa and a substrate CTE in a range from about 0 ppm/° C. to about 120 ppm/° C.; an adhesive comprising a modulus in a range from about 15 MPa to about 100 MPa and a substrate CTE in a range from about 0 ppm/° C. to about 120 ppm/° C. at 15 MPa and decreasing linearly to a range from about 0 ppm/° C. to substrate CTE of about 60 ppm/° C. at 100 MPa; an adhesive comprising a modulus in a range from about 100 MPa to about 500 MPa and a substrate CTE in a range from about 0 ppm/° C. to about 60 ppm/° C. at 100 MPa and decreasing linearly to a range from about 0 ppm/° C. to substrate CTE of about 30 ppm/° C. at 500 MPa; an adhesive comprising a modulus in a range from about 500 MPa to about 1000 MPa and a substrate CTE in a range from about 0 ppm/° C. to about 30 ppm/° C. at 500 MPa and decreasing linearly to a range from about 0 ppm/° C. to substrate CTE of about 15 ppm/° C. at 1000 MPa; and an adhesive comprising a modulus in a range from about 1000 MPa to about 10000 MPa based on the CTE from about 0 ppm/° C. to about 15 ppm/° C.

In other instances, when the glass radius of curvature is less than 400 mm and greater than or equal to about 150 mm the product comprises at least one of: an adhesive comprising a modulus in a range from about 2 MPa to about 5 MPa and a substrate CTE in a range from about 0 ppm/° C. to about 120 ppm/° C.; an adhesive comprising a modulus in a range from about 5 MPa to about 15 MPa and a substrate CTE in a range from about 0 ppm/° C. to about 120 ppm/° C.; an adhesive comprising a modulus in a range from about 15 MPa to about 100 MPa and a substrate CTE in a range from about 0 ppm/° C. to about 120 ppm/° C. at 15 MPa and decreasing linearly to a range from about 0 ppm/° C. to substrate CTE of about 60 ppm/° C. at 100 MPa; an adhesive comprising a modulus in a range from about 100 MPa to about 500 MPa and a substrate CTE in a range from about 0 ppm/° C. to about 60 ppm/° C. at 100 MPa and decreasing linearly to a range from about 0 ppm/° C. to substrate CTE of about 30 ppm/° C. at 500 MPa; an adhesive comprising a modulus in a range from about 500 MPa to about 1000 MPa and a substrate CTE in a range from about 0 ppm/° C. to about 30 ppm/° C. at 500 MPa and decreasing linearly to a range from about 0 ppm/° C. to substrate CTE of about 15 ppm/° C. at 1000 MPa; and an adhesive comprising a modulus in a range from about 1000 MPa to about 10000 MPa based on the CTE from about 0 ppm/° C. to about 15 ppm/° C.

Although metal substrates are discussed herein, the substrate can be made of any suitable material, including metal, (e.g., stainless steel and aluminum) and polymeric materials such as plastic or fiber reinforced composites.

The calculating steps of the methods described herein can be implemented in the context of a machine and an associated software architecture (e.g., ANSYS Mechanical Enterprise mechanical engineering software solution that uses finite element analysis (FEA) for structural analysis using the ANSYS Mechanical interface to model advanced materials in areas such as layered composite materials). The sections below describe representative software architecture(s) and machine (e.g., hardware) architecture(s) that are suitable for use with the disclosed methods.

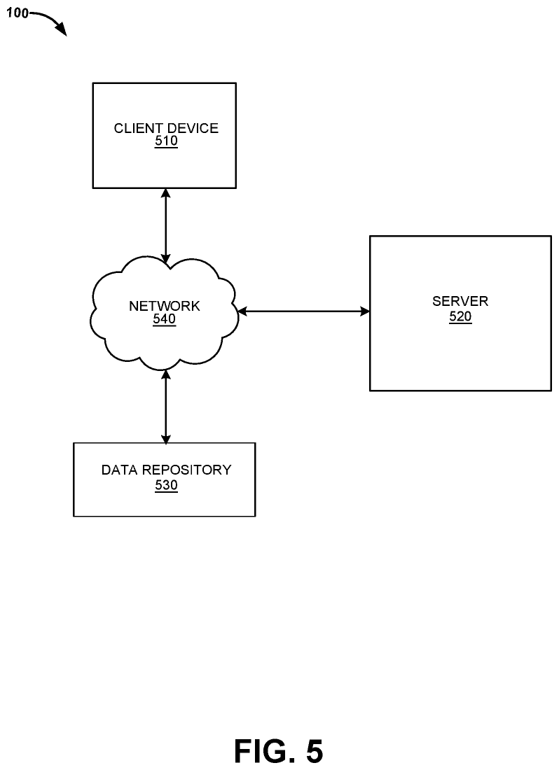

FIG. 5 illustrates an example system 500 in which the calculating steps of the methods described herein can be implemented. As shown, the system 500 includes a client device 510, a server 520, and a data repository 530 communicating with one another over a network 540. The network 540 may include one or more of the internet, an intranet, a local area network, a wide area network, a wired network, a wireless network, and the like.

The system 500 is shown to include a single client device 510, a single server 520, and a single data repository 530. However, the technology described herein may be implemented with multiple client devices, servers, and/or data repositories. Furthermore, the technology is described in FIG. 5 as being implemented in a system 500 that includes the network 540. However, in alternative embodiments, the technology may be implemented using a single machine (which may or may not be connected to a network) or using multiple machines that are connected to each other via a wired or wireless connection that is not a network.

In some examples, the functions of the server 520 may be performed by multiple different machines. In some examples, the data repository 530 may include multiple different machines. In some examples, a single machine performs the functions of both the server 520 and the data repository 530.

The client device 510 may be a laptop computer, a desktop computer, a mobile phone, a tablet computer, a smart watch, a smart speaker device, a smart television, a personal digital assistant (PDA), and the like. The client device 510 may include any device that is used, by an end user, to provide input or receive output.

The data repository 530 stores a plurality of, e.g., physical properties of the cold-formed glass, the metal substrate and/or the adhesive.

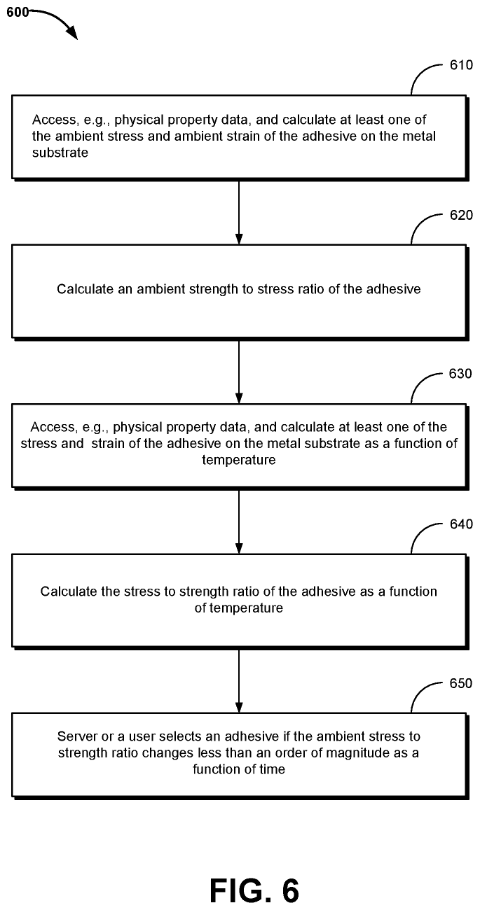

The server 520 stores module 525 that, when executed by the server 520, causes the server 520 to implement all or a portion of the operations of the method 600 described in conjunction with FIG. 6.

FIG. 6 illustrates a flow chart for an example method 600 for selecting an adhesive. The method 600 may be implemented at the server 620 while executing module 625.

At operation 610, the server 520 accesses, e.g., physical property data of the cold-formed glass, the metal substrate and/or the adhesive and calculates at least one of the ambient stress and ambient strain of the adhesive on the metal substrate;

At operation 620, the server 520 calculates an ambient stress to strength ratio of the adhesive.

At operation 630, the server 520 accesses, e.g., physical property data of the cold-formed glass, the metal substrate and/or the adhesive and calculates at least one of the stress and strain of the adhesive on the metal substrate as a function of temperature.

At operation 640, the server 520 calculates the stress to strength ratio of the adhesive as a function of temperature.

At operation 650, the server 520 or a user selects an adhesive if the ambient stress to strength ratio changes less than an order of magnitude as a function of time.

It should be noted that, while the operations 610-650 of the method 600 are specified as being performed in a certain order, in some examples, the operations 610-650 may be performed in a different order. In some cases, one or more of the operations 610-650 may be skipped.

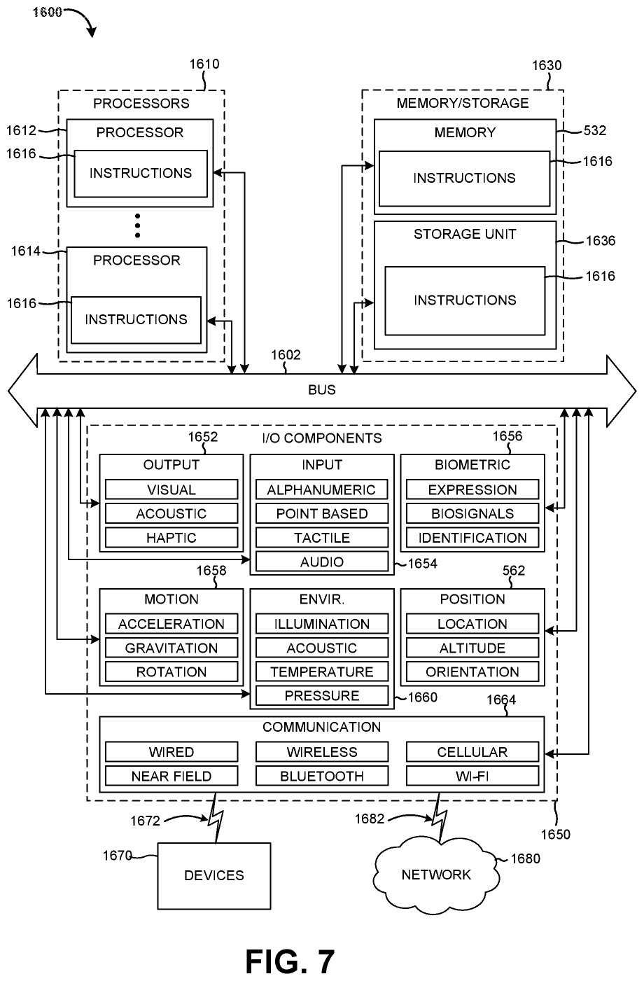

FIG. 7 is a block diagram illustrating components of a machine 1600, according to some example embodiments, able to read instructions from a machine-readable medium (e.g., a machine-readable storage medium) and perform any one or more of the methodologies discussed herein. Specifically, FIG. 7 shows a diagrammatic representation of the machine 1600 in the example form of a computer system, within which instructions 1616 (e.g., software, a program, an application, an applet, an app, or other executable code) for causing the machine 1600 to perform any one or more of the methodologies discussed herein may be executed. The instructions 1616 transform the general, non-programmed machine into a particular machine programmed to carry out the described and illustrated functions in the manner described. In alternative embodiments, the machine 1600 operates as a standalone device or may be coupled (e.g., networked) to other machines. In a networked deployment, the machine 1600 may operate in the capacity of a server machine or a client machine in a server-client network environment, or as a peer machine in a peer-to-peer (or distributed) network environment. The machine 1600 may comprise, but not be limited to, a server computer, a client computer, PC, a tablet computer, a laptop computer, a netbook, a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1616, sequentially or otherwise, that specify actions to be taken by the machine 1600. Further, while only a single machine 1600 is illustrated, the term “machine” shall also be taken to include a collection of machines 1600 that individually or jointly execute the instructions 1616 to perform any one or more of the methodologies discussed herein.

The machine 1600 may include processors 1610, memory/storage 1630, and I/O components 1650, which may be configured to communicate with each other such as via a bus 1602. In an example embodiment, the processors 1610 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1612 and a processor 1614 that may execute the instructions 1616. The term “processor” is intended to include multi-core processors that may comprise two or more independent processors (sometimes referred to as “cores”) that may execute instructions contemporaneously. Although FIG. 7 shows multiple processors 1610, the machine 1600 may include a single processor with a single core, a single prPocessor with multiple cores (e.g., a multi-core processor), multiple processors with a single core, multiple processors with multiples cores, or any combination thereof.

The memory/storage 1630 may include a memory 1632, such as a main memory, or other memory storage, and a storage unit 1636, both accessible to the processors 1610 such as via the bus 1602. The storage unit 1636 and memory 1632 store the instructions 1616 embodying any one or more of the methodologies or functions described herein. The instructions 1616 may also reside, completely or partially, within the memory 1632, within the storage unit 1636, within at least one of the processors 1610 (e.g., within the processor's cache memory), or any suitable combination thereof, during execution thereof by the machine 1600. Accordingly, the memory 1632, the storage unit 1636, and the memory of the processors 1610 are examples of machine-readable media.

As used herein, “machine-readable medium” means a device able to store instructions (e.g., instructions 1616) and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store the instructions 1616. The term “machine-readable medium” shall also be taken to include any medium, or combination of multiple media, that is capable of storing instructions (e.g., instructions 1616) for execution by a machine (e.g., machine 1600), such that the instructions, when executed by one or more processors of the machine (e.g., processors 1610), cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 1650 may include a wide variety of components to receive input, provide output, produce output, transmit information, exchange information, capture measurements, and so on. The specific I/O components 1650 that are included in a particular machine will depend on the type of machine. For example, portable machines such as mobile phones will likely include a touch input device or other such input mechanisms, while a headless server machine will likely not include such a touch input device. It will be appreciated that the I/O components 1650 may include many other components that are not shown in FIG. 7. The I/O components 1650 are grouped according to functionality merely for simplifying the following discussion and the grouping is in no way limiting. In various example embodiments, the I/O components 1650 may include output components 1652 and input components 1654. The output components 1652 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1654 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1650 may include biometric components 1656, motion components 1658, environmental components 1660, or position components 1662, among a wide array of other components. For example, the biometric components 1656 may include components to detect expressions (e.g., hand expressions, facial expressions, vocal expressions, body gestures, or eye tracking), measure biosignals (e.g., blood pressure, heart rate, body temperature, perspiration, or brain waves), measure exercise-related metrics (e.g., distance moved, speed of movement, or time spent exercising) identify a person (e.g., voice identification, retinal identification, facial identification, fingerprint identification, or electroencephalogram based identification), and the like. The motion components 1658 may include acceleration sensor components (e.g., accelerometer), gravitation sensor components, rotation sensor components (e.g., gyroscope), and so forth. The environmental components 1660 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that may provide indications, measurements, or signals corresponding to a surrounding physical environment. The position components 1662 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1650 may include communication components 1664 operable to couple the machine 1600 to a network 1680 or devices 1670 via a coupling 1682 and a coupling 1672, respectively. For example, the communication components 1664 may include a network interface component or other suitable device to interface with the network 1680. In further examples, the communication components 1664 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1670 may be another machine or any of a wide variety of peripheral devices (e.g., a peripheral device coupled via a USB).

Moreover, the communication components 1664 may detect identifiers or include components operable to detect identifiers. For example, the communication components 1664 may include Radio Frequency Identification (RFID) tag reader components, NFC smart tag detection components, optical reader components, or acoustic detection components (e.g., microphones to identify tagged audio signals). In addition, a variety of information may be derived via the communication components 1664, such as location via Internet Protocol (IP) geolocation, location via Wi-Fi® signal triangulation, location via detecting an NFC beacon signal that may indicate a particular location, and so forth.

In various example embodiments, one or more portions of the network 1680 may be an ad hoc network, an intranet, an extranet, a virtual private network (VPN), a local area network (LAN), a wireless LAN (WLAN), a WAN, a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 1680 or a portion of the network 1680 may include a wireless or cellular network and the coupling 1682 may be a Code Division Multiple Access (CDMA) connection, a Global System for Mobile communications (GSM) connection, or another type of cellular or wireless coupling. In this example, the coupling 1682 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 4G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1616 may be transmitted or received over the network 1680 using a transmission medium via a network interface device (e.g., a network interface component included in the communication components 1664) and utilizing any one of a number of well-known transfer protocols (e.g., HTTP). Similarly, the instructions 1616 may be transmitted or received using a transmission medium via the coupling 1672 (e.g., a peer-to-peer coupling) to the devices 1670. The term “transmission medium” shall be taken to include any intangible medium that is capable of storing, encoding, or carrying the instructions 1616 for execution by the machine 1600, and includes digital or analog communications signals or other intangible media to facilitate communication of such software.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

### EXAMPLES

The present invention can be better understood by reference to the following examples which are offered by way of illustration. The present invention is not limited to the examples given herein.

The calculations presented in the Examples that follow were performed using a finite element analysis (FEA) tool to model the stress for an assembly configuration similar to the one shown in FIG. 2, where the adhesive 160 is located between the frame 150 and the glass 140. An example of a suitable FEA tool is the ANSYS Mechanical Enterprise mechanical engineering software tool.

Briefly, the results presented herein were obtained by generating the geometry of the physical part assembly in the FEA software. The physical operating conditions were converted to appropriate modeling abstractions. This includes boundary conditions, material models, etc. After the model is set up, it is run using a computer system, such as the one described in FIG. 7. The results were then interpreted and analyzed to obtain information, such as part deformation, stress, strain, and stress to strength ratio.

The results presented in the following examples demonstrate that adhesive selection can be governed by two important factors, namely, the stress to strength ratio of an adhesive in a given operating temperature range and the material of frame substrate. The results also demonstrate that frame thickness and adhesive thickness are less important factors in the selection of an adhesive.

Although all of the adhesives discussed in the Examples appear to be satisfactory for constructing an assembly configuration that can be used in an automotive interior, one conclusion drawn from the modeled data is that polyurethanes will likely provide the most adequate bonding, followed by silicones/polysiloxanes/silane-modified polymers and epoxies.

### Example 1

Stress modeling was performed for stainless steel and aluminum frames with polyurethane DP604NS available from 3M®, Saint Paul, Minn. at two different temperatures: 95° C. and −40° C. These two temperatures were used because the assembly configurations described herein are likely to encounter such temperature conditions. The maximum principal stress, maximum shear stress, and the maximum von Mises stress values are given in Table 2.

TABLE 2

| | |
|---|---|
| Stainless Steel | Aluminum |

| Stress | 95° C. | −40° C. | 95° C. | −40° C. |
|---|---|---|---|---|
| Max. Princip. (MPa) | 1.3 | 3.2 | 7.4 | 10.4 |
| Max. Shear (MPa) | 0.7 | 1.3 | 2.9 | 4.4 |
| Max. von Mises | 1.2 | 2.3 | 5.2 | 8.0 |
| (MPa) | | | | |

These results show that the stress on the assembly configurations described herein change as a function of material, though the stresses on aluminum are calculated to be higher than the stresses on stainless steel.

### Example 2

Stress modeling was also performed for aluminum frames with polyurethane DP604NS available from 3M®, Saint Paul, Minn.; Scotch-Weld™ Epoxy Adhesive DP125 available from 3M®, Saint Paul, Minn.; and polysiloxane TEROSON RB IX, also known as TEROSTAT MS 9399, available from Loctite®. Again, the stress modeling was performed at two different temperatures: 95° C. and −40° C.

TABLE 3

| | DP604NS | | DP 125 | | MS 9399 | |
|---|---|---|---|---|---|---|
| Stress | 95° C. | −40° C. | 95° C. | −40° C. | 95° C. | −40° C. |
| Max. | 1.1 | 3.2 | 0.2 | 23.1 | 0.5 | 0.9 |
| Princip. | | | | | | |
| (MPa) | | | | | | |
| Max. | 0.7 | 1.4 | 0.1 | 11.3 | 0.2 | 0.4 |
| Shear | | | | | | |
| (MPa) | | | | | | |
| Max. von | 1.2 | 2.4 | 0.2 | 20.2 | 0.3 | 0.6 |
| Mises | | | | | | |
| (MPa) | | | | | | |

These results show that the stresses on the assembly configurations described herein change least dramatically for DP604NS and most dramatically for DP 125, as a function of temperature. very much as a function of temperature.

### Example 3

Stress modeling was performed for aluminum frames with polyurethane DP604NS available from 3M®, Saint Paul, Minn. at two different frame thicknesses, to determine if frame thickness might impact the stress. The maximum principal stress, maximum shear stress, and the maximum von Mises stress values are given in Table 4.

TABLE 4

| Stress | 3 mm | 5 mm |
|---|---|---|
| Max. Princip. (MPa) | 7.8 | 8.5 |
| Max. Shear (MPa) | 3.1 | 3.3 |
| Max. von Mises | 5.6 | 6.0 |
| (MPa) | | |

These results show that the stresses on the assembly configurations described herein do not change very much as a function of frame thickness at 95° C.

### Example 4

Stress modeling was also performed for aluminum frames with polyurethane DP604NS available from 3M®, Saint Paul, Minn. at three different adhesive thicknesses.

TABLE 5

| Stress | 0.6 mm | 1 mm | 2 mm |
|---|---|---|---|
| Max. Princip. (MPa) | 9.1 | 8.5 | 6.7 |
| Max. Shear (MPa) | 3.7 | 3.3 | 2.7 |
| Max. von Mises | 6.6 | 6.0 | 4.9 |
| (MPa) | | | |

These results show that the various stresses on the assembly configurations described herein decrease as a function of adhesive thickness.

The present disclosure provides for the following embodiments, the numbering of which is not to be construed as designating levels of importance:

Embodiment 1 relates to a method for selecting an adhesive for bonding a cold-formed glass to a metal substrate, the method comprising:

calculating at least one of the ambient stress and ambient strain of the adhesive on the metal substrate;
calculating an ambient stress to strength ratio of the adhesive;
calculating at least one of the stress and strain of the adhesive on the metal substrate as a function of temperature;
calculating a stress to strength ratio of the adhesive as a function of temperature; and selecting the adhesive if the ambient stress to strength ratio changes less than an order of magnitude as a function of time.

Embodiment 2 relates to the method of Embodiment 1, wherein the calculating of at least one of the ambient stress and ambient strain of the adhesive on the metal substrate is based on at least one of a thickness of the cold-formed glass, a thickness of the metal substrate, and a thickness of the adhesive.

Embodiment 3 relates to the method of Embodiment 1, wherein the calculating of at least one of the ambient stress and ambient strain of the adhesive on the metal substrate is based on a physical property of at least one of the cold-formed glass, the metal substrate, and the adhesive.

Embodiment 4 relates to the method of Embodiment 3, wherein the physical property is at least one of: the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature (T_{g}) of the adhesive.

Embodiment 5 relates to the method of Embodiment 4, wherein the adhesive is cured such that the difference in the storage modulus (E′) of the material at its lowest operating temperature and highest operating temperature is at about or less than about three orders of magnitude, at about or less than about two orders of magnitude, or at about or less than about one order of magnitude.

Embodiment 6 relates to the method of Embodiment 5, wherein the T_{g} of an adhesive is at about or below about room temperature (e.g., 24° C.), below −10° C., below −20° C. or below −30 C.

Embodiment 7 relates to the method of Embodiment 1, wherein the calculating of at least one of the ambient stress and ambient strain of the adhesive on the metal substrate is based on a bending radius of the cold-formed glass.

Embodiment 8 relates to the method of Embodiment 1, wherein the calculating at least one of the stress and strain of the adhesive on the metal substrate as a function of temperature is based on at least one of a thickness of the cold-formed glass, a thickness of the metal substrate, and a thickness of the adhesive.

Embodiment 9 relates to the method of Embodiment 1, wherein the calculating at least one of the stress and strain of the adhesive on the metal substrate as a function of temperature is based on a physical property of at least one of the cold-formed glass, the metal substrate, and the adhesive.

Embodiment 10 relates to the method of Embodiment 9, wherein the physical property is at least one of: the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature of the adhesive.

Embodiment 11 relates to the method of Embodiment 1, wherein the calculating at least one of the stress and strain of the adhesive on the metal substrate as a function of temperature is based on a bending radius of the cold-formed glass.

Embodiment 12 relates to the method of Embodiment 1, wherein the ambient stress to strength ratio changes from about 3:10 to about 3:100 as a function of time.

Embodiment 13 relates to the method of Embodiment 1, wherein the ambient stress to strength ratio changes from about 3:10 to about 3:100 as a function of a 15 year time period.

Embodiment 14 relates to an adhesive selected using the method of claim 1.

Embodiment 15 relates to the adhesive of Embodiment 14, wherein the adhesive is an intermediate adhesive.

Embodiment 16 relates to the adhesive of Embodiment 14, wherein the adhesive is a polyurethane, a polysiloxane or an epoxy.

Embodiment 17 relates to the adhesive of Embodiment 14, wherein the adhesive is a polyurethane.

Embodiment 18 relates to the adhesive of Embodiment 14, wherein the adhesive is a polysiloxane or a silane-modified polymer.

Embodiment 19 relates to a cold-formed automotive part comprising:

a metal substrate having a first major surface;
a cold-formed glass having a first major surface; and
an adhesive having a first major surface and a second major surface, the adhesive selected using the method of Embodiment 1;
wherein:
the adhesive is located between the metal substrate first major surface and the cold-formed glass first major surface; and
the adhesive bonds the metal substrate first major surface to the cold-formed glass first major surface.

Embodiment 20 relates to the automotive part of Embodiment 19, wherein the adhesive is a polyurethane.

Embodiment 21 relates to a cold-formed product comprising: a structural substrate comprising a curved surface of defined radius of curvature and structural substrate coefficient of thermal expansion (CTE); a cold-formed and curved glass substrate attached to the curved surface with an adhesive, the glass substrate having a glass radius of curvature and comprising a glass substrate CTE, the structural substrate and adhesive forming a structural substrate/adhesive interface and the glass substrate and the adhesive forming a glass substrate/adhesive interface, wherein the glass substrate CTE and the structural substrate CTE differ, wherein the product withstands overlap shear failure as determined by modified test method ASTM D1002-10 at −40° C., 24° C., and 85° C. and tensile failure as determined by ASTM D897 at −40° C., 24° C., and 85° C. at one or both of the structural substrate/adhesive interface and the glass substrate/adhesive interface.

Embodiment 22 relates to the product of Embodiment 21, wherein the glass substrate comprises an ink layer forming an inked surface that is in contact with the adhesive at the glass substrate/adhesive interface.

Embodiment 23 relates to the product of Embodiment 21 or 22, wherein the cold-formed and curved glass substrate comprises a radius of curvature and the structural substrate comprises a radius of curvature, wherein the radius of curvature of the glass substrate and the structural support are within 10% or less of one another.

Embodiment 24 relates to the product of any one of Embodiments 21-23, wherein the radius of curvature of the glass substrate is greater than or equal to about 400 mm, and wherein the product comprises at least one of: the adhesive comprising a modulus in a range from about 0.5 MPa to about 5 MPa and wherein the structural substrate CTE is in a range from about 0 ppm/° C. to about 120 ppm/° C.; the adhesive comprising a modulus in a range from about 5 MPa to about 15 MPa and wherein the structural substrate CTE is in a range from about 0 ppm/° C. to about 120 ppm/° C.; the adhesive comprising a modulus in a range from about 15 MPa to about 100 MPa and wherein the structural substrate CTE is in a range from about 0 ppm/° C. to about 120 ppm/° C. at 15 MPa and decreasing linearly to a range from about 0 ppm/° C. to substrate CTE of about 60 ppm/° C. at 100 MPa; the adhesive comprising a modulus in a range from about 100 MPa to about 500 MPa and wherein the structural substrate CTE is in a range from about 0 ppm/° C. to about 60 ppm/° C. at 100 MPa and decreasing linearly to a range from about 0 ppm/° C. to substrate CTE of about 30 ppm/° C. at 500 MPa; the adhesive comprising a modulus in a range from about 500 MPa to about 1000 MPa and wherein the structural substrate CTE is in a range from about 0 ppm/° C. to about 30 ppm/° C. at 500 MPa and decreasing linearly to a range from about 0 ppm/° C. to substrate CTE of about 15 ppm/° C. at 1000 MPa; and the adhesive comprising a modulus in a range from about 1000 MPa to about 10000 MPa and wherein the structural substrate CTE is from about 0 ppm/° C. to about 15 ppm/° C.

Embodiment 25 relates to the product of any one of Embodiments 21-23, wherein the radius of curvature of the glass substrate is greater than or equal to about 150 mm and less than 400 mm, and wherein the product comprises at least one of: the adhesive comprising a modulus in a range from about 2 MPa to about 5 MPa and wherein the structural substrate CTE is in a range from about 0 ppm/° C. to about 120 ppm/° C.; the adhesive comprising a modulus in a range from about 5 MPa to about 15 MPa and wherein the structural substrate CTE is in a range from about 0 ppm/° C. to about 120 ppm/° C.; the adhesive comprising a modulus in a range from about 15 MPa to about 100 MPa and wherein the structural substrate CTE is in a range from about 0 ppm/° C. to about 120 ppm/° C. at 15 MPa and decreasing linearly to a range from about 0 ppm/° C. to structural substrate CTE of about 60 ppm/° C. at 100 MPa; the adhesive comprising a modulus in a range from about 100 MPa to about 500 MPa and wherein the structural substrate CTE is in a range from about 0 ppm/° C. to about 60 ppm/° C. at 100 MPa and decreasing linearly to a range from about 0 ppm/° C. to structural substrate CTE of about 30 ppm/° C. at 500 MPa; the adhesive comprising a modulus in a range from about 500 MPa to about 1000 MPa and wherein the structural substrate CTE is in a range from about 0 ppm/° C. to about 30 ppm/° C. at 500 MPa and decreasing linearly to a range from about 0 ppm/° C. to a structural substrate CTE of about 15 ppm/° C. at 1000 MPa; and the adhesive comprising a modulus in a range from about 1000 MPa to about 10000 MPa based on a structural CTE from about 0 ppm/° C. to about 15 ppm/° C.

Embodiment 26 relates to the product of any one of Embodiments 21 to 25, wherein the structural substrate is one of a metal, a hybrid of metal, plastic, or fiber reinforced composite.

1. A method for selecting an adhesive for bonding a cold-formed glass to a metal substrate, the method comprising:
calculating at least one of the ambient stress and ambient strain of the adhesive on the metal substrate;
calculating an ambient stress to strength ratio of the adhesive;
calculating at least one of the stress and strain of the adhesive on the metal substrate as a function of temperature;
calculating a stress to strength ratio of the adhesive as a function of temperature; and
selecting the adhesive if the ambient stress to strength ratio changes less than an order of magnitude as a function of time.

2. The method of claim 1, wherein the calculating of at least one of the ambient stress and ambient strain of the adhesive on the metal substrate is based on at least one of a thickness of the cold-formed glass, a thickness of the metal substrate, and a thickness of the adhesive.

3. The method of claim 1, wherein the calculating of at least one of the ambient stress and ambient strain of the adhesive on the metal substrate is based on a physical property of at least one of the cold-formed glass, the metal substrate, and the adhesive, wherein the physical property is at least one of: the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature (T_{g}) of the adhesive.

4. (canceled)

5. The method of claim 3, wherein the Tg is about or below about room temperature such that the difference in the storage modulus (E′) of the material at its lowest operating temperature and highest operating temperature is at about or less than about three orders of magnitude or at about or less than about two orders of magnitude.

6. The method of claim 3, wherein the T_{g} of an adhesive is at or below room temperature.

7. The method of claim 1, wherein the calculating of at least one of the ambient stress and ambient strain of the adhesive on the metal substrate is based on a bending radius of the cold-formed glass.

8. The method of claim 1, wherein the calculating at least one of the stress and strain of the adhesive on the metal substrate as a function of temperature is based on at least one of a thickness of the cold-formed glass, a thickness of the metal substrate, and a thickness of the adhesive.

9. The method of claim 1, wherein the calculating at least one of the stress and strain of the adhesive on the metal substrate as a function of temperature is based on a physical property of at least one of the cold-formed glass, the metal substrate, and the adhesive, wherein the physical property is at least one of: the elasticity, hyper-elasticity or viscoelasticity of the cold-formed glass, the elasticity, hyper-elasticity or viscoelasticity of the metal substrate, the elasticity, hyper-elasticity or viscoelasticity of the adhesive, and the glass transition temperature of the adhesive.

10. (canceled)

11. The method of claim 1, wherein the calculating at least one of the stress and strain of the adhesive on the metal substrate as a function of temperature is based on a bending radius of the cold-formed glass.

12. (canceled)

13. The method of claim 1, wherein the ambient stress to strength ratio changes from about 3:10 to about 3:100 as a function of a 15 year time period.

14. (canceled)

15. (canceled)

16. The adhesive of claim 1, wherein the adhesive is a polyurethane, a polysiloxane or an epoxy.

17. (canceled)

18. The adhesive of claim 1, wherein the adhesive is a polysiloxane or a silane-modified polymer.

19. An automotive part comprising:
a metal substrate having a first major surface;
a cold-formed glass having a first major surface; and
an adhesive having a first major surface and a second major surface, the adhesive selected using the method of claim 1;
wherein:
the adhesive is located between the metal substrate first major surface and the cold-formed glass first major surface; and
the adhesive bonds the metal substrate first major surface to the cold-formed glass first major surface.

20. The automotive part of claim 19, wherein the adhesive is a polyurethane.

21. A cold-formed product comprising:
a structural substrate comprising a curved surface and structural substrate coefficient of thermal expansion (CTE);
a cold-formed and curved glass substrate attached to the curved surface with an adhesive, the glass substrate comprising a glass substrate CTE, the structural substrate and adhesive forming a structural substrate/adhesive interface and the glass substrate and the adhesive forming a glass substrate/adhesive interface,
wherein the glass substrate CTE and the structural substrate CTE differ,
wherein the product withstands overlap shear failure as determined by modified test method ASTM D1002-10 at −40° C., 24° C., and 85° C. and tensile failure as determined by ASTM D897 at −40° C., 24° C., and 85° C. at one or both of the structural substrate/adhesive interface and the glass substrate/adhesive interface.

22. The product of claim 21, wherein the glass substrate comprises an ink layer forming an inked surface that is in contact with the adhesive at the glass substrate/adhesive interface.

23. The product of claim 21, wherein the cold-formed and curved glass substrate comprises a radius of curvature and the structural substrate comprises a radius of curvature, wherein the radius of curvature of the glass substrate and the structural support are within 10% or less of one another.

24. The product of claim 21, wherein the glass substrate radius of curvature is greater than or equal to about 400 mm, and wherein the product comprises at least one of:
the adhesive comprising a modulus in a range from about 0.5 MPa to about 5 MPa and wherein the structural substrate CTE is in a range from about 0 ppm/° C. to about 120 ppm/° C.; the adhesive comprising a modulus in a range from about 5 MPa to about 15 MPa and wherein the structural substrate CTE is in a range from about 0 ppm/° C. to about 120 ppm/° C.;
the adhesive comprising a modulus in a range from about 15 MPa to about 100 MPa and wherein the structural substrate CTE is in a range from about 0 ppm/° C. to about 120 ppm/° C. at 15 MPa and decreasing linearly to a range from about 0 ppm/° C. to substrate CTE of about 60 ppm/° C. at 100 MPa;
the adhesive comprising a modulus in a range from about 100 MPa to about 500 MPa and wherein the structural substrate CTE is in a range from about 0 ppm/° C. to about 60 ppm/° C. at 100 MPa and decreasing linearly to a range from about 0 ppm/° C. to substrate CTE of about 3 0 ppm/° C. at 500 MPa;
the adhesive comprising a modulus in a range from about 500 MPa to about 1000 MPa and wherein the structural substrate CTE is in a range from about 0 ppm/° C. to about 30 ppm/° C. at 500 MPa and decreasing linearly to a range from about 0 ppm/° C. to substrate CTE of about 15 ppm/° C. at 1000 MPa; and
the adhesive comprising a modulus in a range from about 1000 MPa to about 10000 MPa and wherein the structural substrate CTE is from about 0 ppm/° C. to about 15 ppm/° C.

25. The product of claim 21, wherein the glass substrate radius of curvature is greater than or equal to about 150 mm and less than 400 mm, and wherein the product comprises at least one of:
the adhesive comprising a modulus in a range from about 2 MPa to about 5 MPa and wherein the structural substrate CTE is in a range from about 0 ppm/° C. to about 120 ppm/° C.;
the adhesive comprising a modulus in a range from about 5 MPa to about 15 MPa and wherein the structural substrate CTE is in a range from about 0 ppm/° C. to about 120 ppm/° C.;
the adhesive comprising a modulus in a range from about 15 MPa to about 100 MPa and wherein the structural substrate CTE is in a range from about 0 ppm/° C. to about 120 ppm/° C. at 15 MPa and decreasing linearly to a range from about 0 ppm/° C. to structural substrate CTE of about 60 ppm/° C. at 100 MPa;
the adhesive comprising a modulus in a range from about 100 MPa to about 500 MPa and wherein the structural substrate CTE is in a range from about 0 ppm/° C. to about 60 ppm/° C. at 100 MPa and decreasing linearly to a range from about 0 ppm/° C. to structural substrate CTE of about 3 0 ppm/° C. at 500 MPa;
the adhesive comprising a modulus in a range from about 500 MPa to about 1000 MPa and wherein the structural substrate CTE is in a range from about 0 ppm/° C. to about 30 ppm/° C. at 500 MPa and decreasing linearly to a range from about 0 ppm/° C. to a structural substrate CTE of about 15 ppm/° C. at 1000 MPa; and
the adhesive comprising a modulus in a range from about 1000 MPa to about 10000 MPa based on a structural CTE from about 0 ppm/° C. to about 15 ppm/° C.

26. The product of claim 21, wherein the structural substrate is one of a metal, a hybrid of metal, plastic, or fiber reinforced composite.
